(19) United States
(12) Patent Application Publication
(10) Pub. No.: US 2023/0293821 A1
(43) Pub. Date: Sep. 21, 2023

(54) DOSE SETTING SENSOR ASSEMBLY WITH ALGORITHMIC AUTO CALIBRATION

(71) Applicant: Novo Nordisk A/S, Bagsvaerd (DK)

(72) Inventors: Henrik Bengtsson, Taastrup (DK); Zeinab Mahmoudi, Birkeroed (DK)

(21) Appl. No.: 18/014,566

(22) PCT Filed: Jul. 5, 2021
(86) PCT No.: PCT/EP2021/068517

§ 371 Date: Jan. 5, 2023

(30) Foreign Application Priority Data
Jul. 8, 2020 (EP) 20184661.5

(51) Int. Cl. A61M 5/315

(52) U.S. Cl. CPC A61M 5/3155; A61M 5/31515; A61M 5/31556; A61M 2205/52

(57) ABSTRACT

Drug delivery system comprising a dose setting member adapted to incrementally rotate in a first direction to set a dose, and incrementally rotate in an opposed second direction to reduce a set dose, the dose setting member having a rotational slack in each incremental rotational position. The system further comprises a rotary sensor adapted to detect the amount of rotation of the dose setting member during dose setting. Processor means is adapted to detect a first or second dose setting pattern when the final dose was set by rotating the setting member in the first respectively the second direction. When a first / second dose setting pattern is detected the detected amount of rotation is used to calculate a corrected amount of rotation using a first/ second algorithm compensating for a slack-induced error generated corresponding to the first/ second dose setting pattern.

The present invention generally relates to medical devices and systems for which efficient and reliable detection of set dose amounts is relevant.

### BACKGROUND OF THE INVENTION

In the disclosure of the present invention reference is mostly made to drug delivery devices used e.g. in the treatment of diabetes by subcutaneous delivery of insulin, however, this is only an exemplary use of the present invention.

Drug delivery devices for subcutaneous injections have greatly improved the lives of patients who must self-administer drugs and biological agents. Such drug delivery devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections. A common type of drug delivery devices allows a user to set a desired dose size for the drug to be delivered. For a typical mechanical device the dose setting means is in the form of a rotatable dose setting or dial member allowing the user to set (or “dial”) the desired dose size which is then subsequently expelled from the device.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the notebook system requires that the patient remembers to log each injection, if the logged information is to have any value in the treatment of the patient’s disease. A missing or erroneous record in the log results in a misleading picture of the injection history and thus a misleading basis for the medical personnel’s decision making with respect to future medication. Accordingly, it may be desirable to automate the logging of injection information from medication delivery systems.

Though some injection devices integrate this monitoring/acquisition mechanism into the device itself, e.g. as disclosed in US 2009/0318865 and WO 2010/052275, most devices of today are without it. The most widely used devices are purely mechanical devices being either durable or prefilled. The latter devices are to be discarded after being emptied and so inexpensive that it is not cost-effective to build-in electronic data acquisition functionality in the device it-self. Addressing this problem a number of solutions have been proposed which would help a user to generate, collect and distribute data indicative of the use of a given medical device.

For example, WO 2014/037331 describes in a first embodiment an electronic supplementary device (also named “add-on module” or “add-on device”) adapted to be releasably attached to a drug delivery device of the pen type. The device includes a camera and is configured to perform optical character recognition (OCR) on captured images from a rotating scale drum visible through a dosage window on the drug delivery device, thereby to determine a dose of medicament that has been dialled into the drug delivery device. WO 2014/020008 discloses an electronic supplementary device adapted to be releasably attached to a drug delivery device of the pen type. The device includes a camera and is configured to determine scale drum values based on OCR. To properly determine the size of an expelled dose the supplementary device further comprises additional electromechanical sensor means to determine whether a dose size is set, corrected, or delivered. As appears, this concept requires the add-on device to “interact” with the drug delivery device via both a camera and subsequent image processing, this adding to complexity, reliability, and costs.

A further external device for a pen device is shown in WO 2014/161952.

WO 2019/057911 discloses an add-on dose logging device for a pen-formed drug delivery device, comprising sensor means adapted to capture an amount of rotation of a magnetic member arranged in the drug delivery device, the amount of rotation of the magnetic member corresponding to the amount of drug expelled from a reservoir by the drug delivery device. As appears, this concept requires the drug delivery device to be modified and provided with an integrated magnet.

WO 2020/110124 and US 2017/0182258 disclose drug delivery systems comprising dose logging means adapted to detect and identify specific events during handling and operation of a pen-type drug delivery device. More specifically, the systems are adapted to detect a first “click” event indicative of a dose being increased with an increment, and to detect a second “click” event indicative of a dose being decreased with an increment, this allowing the dose logging means to “count” up respectively down the number of increments being dialled by the rotational dose setting member. To compensate for incorrect event detection the dose logging means may be provided with means allowing a user to adjust the counted dose size to correspond to the actually set dose size.

Irrespective of the specific nature of the sensor means, dose logging circuitry in general rely on the determination of an amount of rotation for a given component in a drug delivery device, the amount of rotation corresponding to the size of an expelled dose of drug for the given drug delivery device.

Having regard to the above, it is an object of the present invention to provide devices, assemblies and methods allowing efficient, reliable, and precise capturing of set dose amounts based on the determination of the amount of rotation of an indicator component. The devices and assemblies may be external to the drug delivery device per se, e.g. relate to add-on devices adapted to be releasably mounted on a drug delivery device, the indicator component interacting with the drug delivery device in a simple and reliable way. Alternatively, the indicator component and additional capturing components may be formed integrally with a given drug delivery device.

### DISCLOSURE OF THE INVENTION

In the disclosure of the present invention, embodiments and aspects will be described which will address one or more of the above objects or which will address objects apparent from the below disclosure as well as from the description of exemplary embodiments.

The present invention is based on the realization that measuring the size of a set dose amount of drug to subsequently be out-dosed, e.g. units of insulin, may be influenced by which component of a given dose setting mechanism that is actually used for the measurement. In a typical dose setting and expelling mechanism a number of components are rotated to incrementally set a desired dose to be expelled, typically starting with a dose setting member being rotated by the user in order to set a rachet mechanism in a given desired rotational position. When the set dose is to be expelled, e.g. by releasing a strained spring mechanism, a number of components are rotated to ultimately result in the piston in a drug-filled cartridge being moved axially in a distal direction.

However, slack and small variations due to production tolerances typically add up through the mechanism and increase inaccuracy of measurement.

For example, when fitting an add-on measuring device on an existing drug delivery device for injection, such an add-on system typically has to measure the change in position of a component which is “at the beginning” of the chain of interacting components. Dose setting adjustment mechanisms are often designed such that the user can dial up a dose size from 0 to a desired dose size, in which case a component is turned a number of “clicks” in a ratchet mechanism.

Each “click” corresponds to a given volume of out-dosing and occurs each time the component is rotated a specific number of degrees. In such ratchet mechanisms, some play or slack between interacting components are necessary to prevent jamming.

This means that with each interacting pair of components, a little slack must be ensured even in worst-case combinations of production tolerance variations, such that even tight- fit combination production tolerances will result in a little play between the interacting parts. This again means that opposite worst-case combinations will have significantly more play. Often dialing mechanisms involve interactions between 4-6 parts, that form a chain of mechanical parts and the slack between each interacting pair of components adds up to a total slack or play in the dialing mechanism. A further 5-10 interacting components may be involved in the actual out-dosing, adding to the chain of inaccuracy and slack. Since such devices are required to be able to expel a volume with given tolerances, the total slack has to be kept lower than about a third of a unit. Production cost increase significantly with reduced production tolerances and thus devices are mostly designed with maximum allowable slack ensuring performance requirements to be met. Thus, a total slack of up to plus/minus about a quarter of a unit quite normal for such devices.

However, most drug delivery devices on the market today allows the user to adjust a set dose. Correspondingly, such dose setting mechanisms have to be designed such that an already dialed dose size, i.e. an already dialed number of “clicks” in the ratchet mechanism, can be dialed back to a lower dose setting, in case the user accidently dials too far or re-decides to take a lower dose.

This means that after having dialed up and thereby having absorbed all slack “to one side”, all interacting parts move from engagement in “one side” to the opposite side, before mechanism actually dials back in the ratchet.

Correspondingly, the input dial, i.e. the component actually gripped by the user, may have to pick up twice the slack (from plus to minus), which is not an issue during normal us, since the user in most cases will not notice the “silent” slack when the dialing direction is reversed. Furthermore, some mechanisms are designed such that additional slack is introduced by a built-in mechanism to disengage or loosen the ratchet mechanism during normal operation to allow dialing backwards.

The above-described additional slack related to dial-down operation mostly affects the input dial component as the further down through the chain of interacting components, the lesser the components will be affected. The actual dose to be expelled may not be influenced at all. However, in relation to an add-on measuring device, measuring the start- and end-positions of a component close to the actual dial-input component, this may present a problem since the start-position of this component may differ by up to almost a full unit depending on the user having dialed up or down to reach the set dose at dose release. Even with a very high accuracy of measurement, it may be difficult to determine what the actually set dose was and consequently what was out-dosed with an accuracy better than plus/minus one unit, which in some cases may not fulfil the authority requirements for such devices.

Thus, having identified a root cause for potential lack of accuracy for a dose logging arrangement relying on determination of rotational movement of a component “close to the dial input member”, an identified problem to be solved is to provide a solution for an add-on device that reduces or eliminates the measuring systems sensitivity to the slack experienced by the component on which measurements are performed between dial-up and dial-down performed by the user. Indeed, such a solution could also be implemented for an integrated dose logging arrangement utilizing the same indicator component.

Thus, in a first general aspect of the invention a drug delivery assembly is provided, comprising a housing defining a reference axis for rotation, a drug reservoir or means for receiving a drug reservoir, and drug expelling means. The drug expelling means comprises a dose setting member adapted to (i) incrementally rotate in a first direction to set a dose, and (ii) incrementally rotate in an opposed second direction to reduce a set dose, the dose setting member having a rotational slack in each incremental rotational position, and an actuation member adapted to cause a final set dose amount to be expelled. The drug delivery assembly further comprises a rotary sensor adapted to detect the amount of rotation of the dose setting member relative to the housing during dose setting, as well as processor means adapted to detect a first dose setting pattern when the final dose was set by rotating the setting member in the first direction, detect a second dose setting pattern when the final dose was set by rotating the setting member in the second direction, when a first dose setting pattern is detected: based on the detected amount of rotation calculating a corrected amount of rotation using a first algorithm compensating for a slack-induced error generated corresponding to the first dose setting pattern, and when a second dose setting pattern is detected: based on the detected amount of rotation calculating a corrected amount of rotation using a second algorithm compensating for a slack-induced error generated corresponding to the second dose setting pattern.

By this arrangement it is ensured that the influence of slack in a drug delivery device dose setting mechanism is reduced or eliminated for a dose measuring system relying on a rotary sensor adapted to detect the amount of rotation of a dose setting member forming part of the dose setting mechanism.

Calculated amounts of rotation and/or thereto related dose data may be stored in processor-controlled memory to create a dose log. Dose data may subsequently be transmitted to an external receiver.

The drug expelling means may comprise a drive spring for expelling a set amount of drug from the drug reservoir, the dose setting member being adapted to (i) incrementally rotate in a first direction to set a dose and strain the drive spring correspondingly, and (ii) incrementally rotate in an opposed second direction to reduce a set dose and unstrain the drive spring correspondingly, wherein the actuation member is a release member adapted to release the strained drive spring to expel a final set dose amount.

For a detected first dose pattern the processor means may be adapted to detect a dose setting pause between two consecutive dose setting rotations in the first direction, and when one or more dose setting pauses are detected: based on the detected amount of rotation calculating a corrected amount of rotation using a third algorithm compensating for a slack-induced error generated corresponding to the first dose setting pattern when one or more dose setting pauses have been detected.

The drug expelling means may be provided with an over-torque mechanism allowing the dose setting member to be rotated further in the first direction when a predetermined maximum dose has been set, the processor means being adapted to detect an over-torque condition and calculate the amount of rotation of the dose setting member relative to the housing corresponding to the set maximum dose.

In an exemplary embodiment the drug delivery system is in the form of an assembly comprising a drug delivery device and an add-on device adapted to be releasably mounted on the drug delivery device. In such a system the drug delivery device comprises the housing, the drug reservoir, or the means for receiving a drug reservoir, and the drug expelling means. The add-on device comprises the rotary sensor, and the processor means.

In a specific embodiment the add-on device further comprises an add-on housing being releasably attachable to the drug delivery device housing, an add-on dose setting member, and an add-on release member axially moveable relative to the add-on dose setting member between a dose setting state and a dose expelling state. The add-on dose setting member is adapted to non-rotationally engage the dose setting member, the rotary sensor is adapted to detect the amount of rotation of the add-on dose setting member relative to the add-on housing during dose setting, and when in a mounted state, the add-on release member is adapted to release the release member when moved from the dose setting state to the dose expelling state.

The rotary sensor may be de-activated when the add-on release member is moved from the dose setting state to the dose expelling state.

The above-described drug delivery system may be provided with drug expelling means comprising a piston rod adapted to engage and axially displace a piston in a loaded cartridge in a distal direction to thereby expel a dose of drug from the cartridge, and a drive member coupled directly or indirectly to the piston rod.

The drug expelling means may comprise a drive spring coupled to the drive member, with the release member being adapted to release the strained drive spring to rotate the drive member to expel the set dose amount. Alternatively, the drug expelling means may comprise an actuation button being axially and proximally extended during dose setting and subsequently driving the expelling means when being moved distally by the user to expel an amount of drug corresponding to a set dose size.

In a further aspect of the invention an add-on device adapted to be releasably mounted on a drug delivery device is provided. The drug delivery device comprises a device housing defining a reference axis for rotation, a drug reservoir or means for receiving a drug reservoir, and drug expelling means. The drug expelling means comprises a device dose setting member adapted to (i) incrementally rotate in a first direction to set a dose, and (ii) incrementally rotate in an opposed second direction to reduce a set dose, the dose setting member having a rotational slack in each incremental rotational position, and a device actuation member adapted to cause a final set dose amount to be expelled. Tthe add-on device comprises an add-on housing being releasably attachable to the device housing, an add-on dose setting member adapted to non-rotationally engage the device dose setting member, an add-on actuation member axially moveable relative to the add-on dose setting member between a dose setting state and a dose expelling state, the add-on actuation member being adapted to, when in a mounted state, actuate the device actuation member when moved from the dose setting state to the dose expelling state, a rotary sensor adapted to detect the amount of rotation of the add-on dose setting member relative to the add-on housing during dose setting, and processor means. The processor means is adapted to detect a first dose setting pattern when the final dose was set by rotating the add-on dose setting member in the first direction, and detect a second dose setting pattern when the final dose was set by rotating the add-on dose setting member in the second direction. When a first dose setting pattern is detected: based on the detected amount of rotation calculating a corrected amount of rotation using a first algorithm compensating for a slack-induced error generated corresponding to the first dose setting pattern, and when a second dose setting pattern is detected: based on the detected amount of rotation calculating a corrected amount of rotation using a second algorithm compensating for a slack-induced error generated corresponding to the second dose setting pattern. The add-on dose setting member is adapted to non-rotationally engage the dose setting member, the rotary sensor is adapted to detect the amount of rotation of the add-on dose setting member relative to the add-on housing during dose setting, and when in a mounted state, the add-on actuation member is adapted to actuate the device actuation member when moved from the dose setting state to the dose expelling state.

The drug expelling means may further comprise a drive spring for expelling a set amount of drug from the drug reservoir, the dose setting member being adapted to (i) incrementally rotate in a first direction to set a dose and strain the drive spring correspondingly, and (ii) incrementally rotate in an opposed second direction to reduce a set dose and unstrain the drive spring correspondingly, wherein the actuation member is a release member adapted to release the strained drive spring to expel a final set dose amount. Alternatively the expelling mechanism may be fully manual in which case the dose member and the actuation button move proximally during dose setting corresponding to the set dose size, and then is moved distally by the user to expel the set dose. To accommodate such a pen device design the add-on device may comprise a first portion adapted to be mounted on the pen device housing, a second portion adapted to be mounted on the pen actuation button, the second portion being axially guided relatively to the first portion.

The rotary sensor of the above-described system or add-on device may be adapted to detect the amount of rotation of the dose setting member relative to the housing during dose setting with a rotational resolution of at least twice the number of increments for a full revolution of the dose setting member. For example, for a drug delivery system adapted to set a dose with 20 increments for one full revolution of the dose setting member, each increment corresponds to a rotational movement of 18 degrees, such a rotary sensor correspondingly having a rotational resolution of 9 degrees or less. For a drug delivery system adapted to set a dose with 24 increments for one full revolution of the dose setting member, each increment corresponds to a rotational movement of 15 degrees, the rotary sensor correspondingly having a rotational resolution of 7.5 degrees or less. In exemplary embodiments having a dose setting mechanism with e.g. 20 or 24 increments for one full turn of the dose setting member, the rotational sensor resolution may be 5 degrees or less, 3 degrees or less, or 1 degree or less.

The rotary sensor may be in the form of a galvanic rotary encoder comprising a circular array of encoder segments. Alternatively the rotary sensor may comprise magnetometers adapted to measure a magnetic field from a moving (rotating) magnet.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described module could also be used to create logs for other types of drug, e.g. growth hormone.

### BRIEF DESCRIPTION OF THE DRAWINGS

In the following embodiments of the invention will be described with reference to the drawings, wherein

FIG. 1A shows a pen device,

FIG. 1B shows the pen device of FIG. 1A with the pen cap removed,

FIG. 2 shows in an exploded view the components of the pen device of FIG. 1A,

FIGS. 3A and 3B show in sectional views an expelling mechanism in two states,

FIGS. 4A and 4B show a schematic representation of an add-on device and a drug delivery device in a non-assembled respectively assembled state,

FIGS. 5A and 5B respectively show an exemplary drug delivery device and a corresponding add-on device adapted to be mounted thereon as shown in FIGS. 4A and 4B,

FIGS. 5C and 5D show in a partial cut-away view the add-on device of FIG. 5B in a dose setting respectively dose expelling state,

FIGS. 6A and 6B show a dialled number of degrees measured in ‘regret’ versus ‘no regret’ scenarios,

FIG. 7 shows distributions of errors in degree in dialling of doses,

FIG. 8 shows a high-level description of an Algorithmic Auto Calibration method,

FIG. 9 shows encoder data with peaks and dips indicators extracted for over-torque detection when a user dials and starts to over-torque,

FIG. 10 shows a first dial event sequence,

FIG. 11 shows a second dial event sequence,

FIG. 12 shows components of a regret dose setting,

FIG. 13 shows regression of actual error versus estimated regret play,

FIG. 14 shows estimated coefficients of β₀ and β₁,

FIG. 15 shows distribution of drop in non-regret cases,

FIG. 16 shows error distribution for dose events with regret,

FIG. 17 shows distribution of regret errors in test data,

FIG. 18 shows dial event with rest,

FIG. 19 shows dial event with two middle rests and final rest,

FIG. 20 shows position of rest points with respect to the unit markers on a scale drum, and

FIG. 21 shows overview of the algorithmic auto calibration signal processing of the encoder signals from a rotational encoder in an add-on unit for an injection device.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

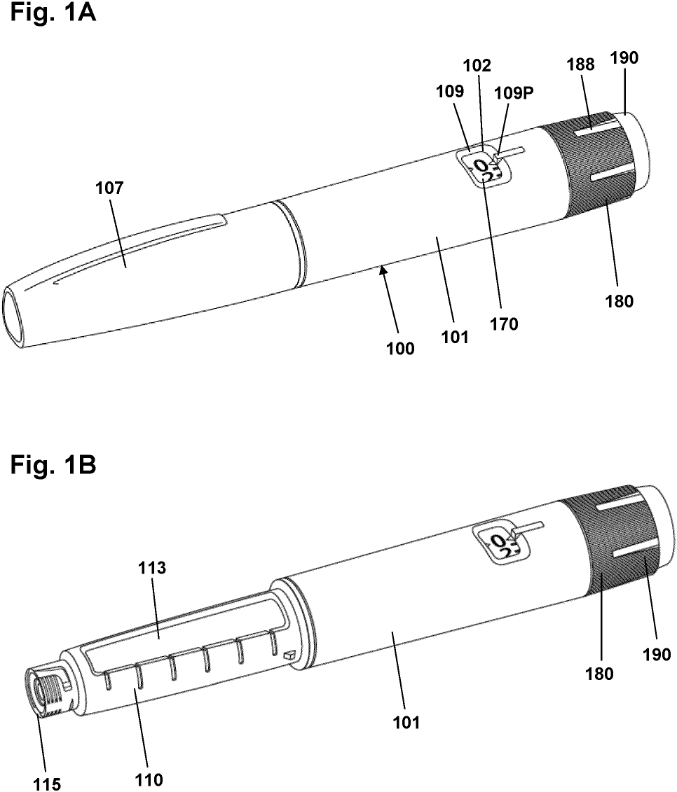

Before turning to embodiments of the present invention per se, an example of a prefilled drug delivery will be described, such a device providing the basis for the exemplary embodiments of the present invention. Although the pen-formed drug delivery device 100 shown in FIGS. 1-3 may represent a “generic” drug delivery device, the actually shown device is a FlexTouch® prefilled drug delivery pen device as manufactured and sold by Novo Nordisk A/S, Bagsværd, Denmark.

The pen device 100 comprises a cap part 107 and a main part having a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion, the cartridge holder having openings allowing a portion of the cartridge to be inspected as well as distal coupling means 115 allowing a needle assembly to be releasably mounted. The cartridge is provided with a piston driven by a piston rod forming part of the expelling mechanism and may for example contain an insulin, GLP-1, or growth hormone formulation. A proximal-most rotatable dose setting member 180 with a number of axially oriented grooves 188 serves to manually set a desired dose of drug shown in display window 102 and which can then be expelled when the button 190 is actuated. As will be apparent from the below description, the shown axially oriented grooves 188 may be termed “drive grooves”. The dose setting member 180 has a generally cylindrical outer surface 181 (i.e. the dose setting member may be slightly tapered) which in the shown embodiment is textured by comprising a plurality of axially oriented fine grooves to improve finger grip during dose setting. The window is in the form of an opening in the housing surrounded by a chamfered edge portion 109 and a dose pointer 109P, the window allowing a portion of a helically rotatable indicator member 170 (scale drum) to be observed. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button move proximally during dose setting corresponding to the set dose size, and then is moved distally by the user to expel the set dose, e.g. as in a FlexPen® manufactured and sold by Novo Nordisk A/S.

Although FIG. 1 shows a drug delivery device of the prefilled type, i.e. it is supplied with a premounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

As the invention relates to electronic circuitry adapted to interact with a drug delivery device, an exemplary embodiment of such a device will be described for better understanding of the invention.

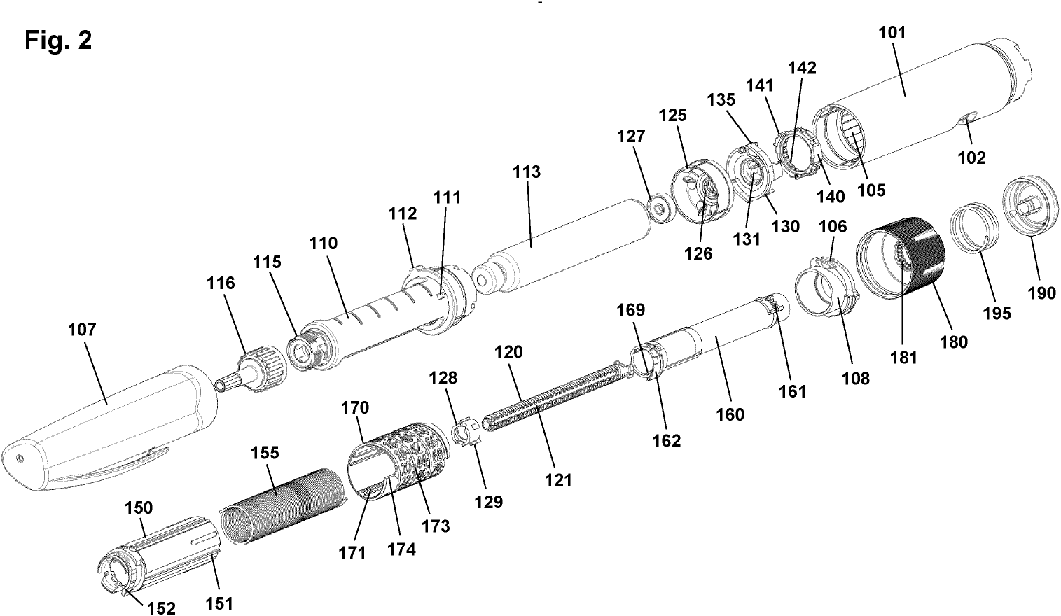

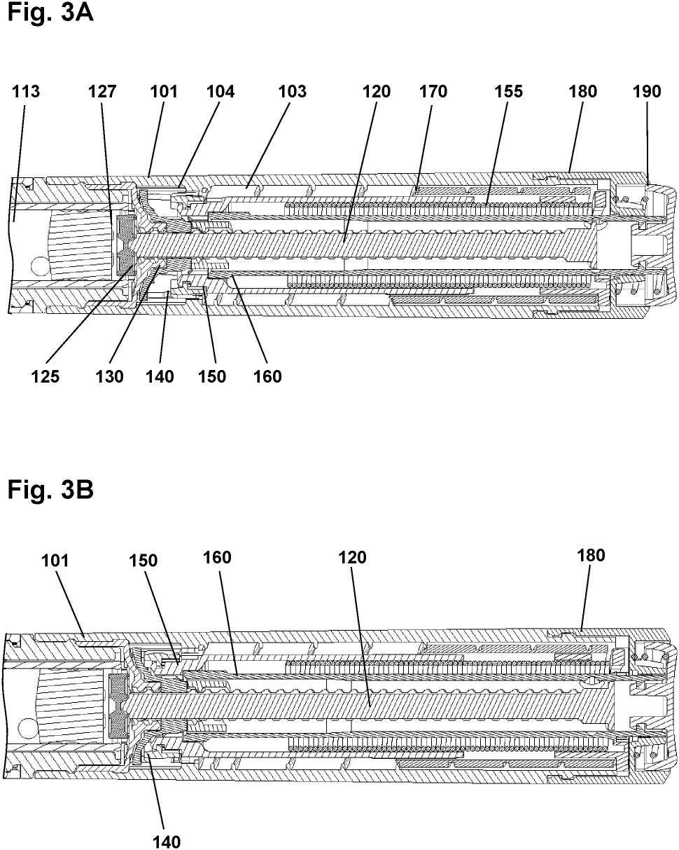

FIG. 2 shows an exploded view of the pen-formed drug delivery device 100 shown in FIG. 1. More specifically, the pen comprises a tubular housing 101 with a window opening 102 and onto which a cartridge holder 110 is fixedly mounted, a drug-filled cartridge 113 being arranged in the cartridge holder. The cartridge holder is provided with distal coupling means 115 allowing a needle assembly 116 to be releasable mounted, proximal coupling means in the form of two opposed protrusions 111 allowing a cap 107 to be releasable mounted covering the cartridge holder and a mounted needle assembly, as well as a protrusion 112 preventing the pen from rolling on e.g. a table top. In the housing distal end a nut element 125 is fixedly mounted, the nut element comprising a central threaded bore 126, and in the housing proximal end a spring base member 108 with a central opening is fixedly mounted. A drive system comprises a threaded piston rod 120 having two opposed longitudinal grooves and being received in the nut element threaded bore, a ring-formed piston rod drive element 130 rotationally arranged in the housing, and a ring-formed clutch element 140 which is in rotational engagement with the drive element (see below), the engagement allowing axial movement of the clutch element. The clutch element is provided with outer spline elements 141 adapted to engage corresponding splines 104 (see FIG. 3B) on the housing inner surface, this allowing the clutch element to be moved between a rotationally locked proximal position, in which the splines are in engagement, and a rotationally free distal position in which the splines are out of engagement. As just mentioned, in both positions the clutch element is rotationally locked to the drive element. The drive element comprises a central bore with two opposed protrusions 131 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 135 adapted to engage corresponding ratchet teeth 105 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3A and 3B.

On the piston rod an end-of-content (EOC) member 128 is threadedly mounted and on the distal end a washer 127 is rotationally mounted. The EOC member comprises a pair of opposed radial projections 129 for engagement with the reset tube (see below).

The dial system comprises a ratchet tube 150, a reset tube 160, a scale drum 170 with an outer helically arranged pattern forming a row of dose indicia, a user-operated dial member 180 for setting a dose of drug to be expelled, a release button 190 and a torque spring 155 (see FIG. 3). The dial member is provided with a circumferential inner teeth structure 181 engaging a number of corresponding outer teeth 161 arranged on the reset tube, this providing a dial coupling which is in an engaged state when the reset tube is in a proximal position during dose setting and in a disengaged state when the reset tube is moved distally during expelling of a dose. The reset tube is mounted axially locked inside the ratchet tube but is allowed to rotate a few degrees (see below). The reset tube comprises on its inner surface two opposed longitudinal grooves 169 adapted to engage the radial projections 129 of the EOC member, whereby the EOC can be rotated by the reset tube but is allowed to move axially. The clutch element is mounted axially locked on the outer distal end portion of the ratchet tube 150, this providing that the ratchet tube can be moved axially in and out of rotational engagement with the housing via the clutch element. The dial member 180 is mounted axially locked but rotationally free on the housing proximal end, the dial ring being under normal operation rotationally locked to the reset tube (see below), whereby rotation of the dial ring results in a corresponding rotation of the reset tube 160 and thereby the ratchet tube. The release button 190 is axially locked to the reset tube but is free to rotate. A return spring 195 provides a proximally directed force on the button and the thereto mounted reset tube. The scale drum 170 is arranged in the circumferential space between the ratchet tube and the housing, the drum being rotationally locked to the ratchet tube via cooperating longitudinal splines 151, 171 and being in rotational threaded engagement with the inner surface of the housing via cooperating thread structures 103, 173, whereby the row of numerals passes the window opening 102 in the housing when the drum is rotated relative to the housing by the ratchet tube. The torque spring is arranged in the circumferential space between the ratchet tube and the reset tube and is at its proximal end secured to the spring base member 108 and at its distal end to the ratchet tube, whereby the spring is strained when the ratchet tube is rotated relative to the housing by rotation of the dial member. A ratchet mechanism with a flexible ratchet arm 152 is provided between the ratchet tube and the clutch element, the latter being provided with an inner circumferential teeth structures 142, each tooth providing a ratchet stop such that the ratchet tube is held in the position to which it is rotated by a user via the reset tube when a dose is set. In order to allow a set dose to be reduced a ratchet release mechanism 162 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system, and a dial system, this as described above. During dose setting the dial mechanism rotates and the torsion spring is loaded. The dose mechanism is locked to the housing and cannot move. When the push button is pushed down, the dose mechanism is released from the housing and due to the engagement to the dial system the torsion spring will now rotate back the dial system to the starting point and rotate the dose system along with it.

The central part of the dose mechanism is the piston rod 120, the actual displacement of the plunger being performed by the piston rod. During dose delivery, the piston rod is rotated by the drive element 130 and due to the threaded interaction with the nut element 125 which is fixed to the housing, the piston rod moves forward in the distal direction. Between the rubber piston and the piston rod, the piston washer 127 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 134 which prevent the drive element from rotating clockwise (seen from the push button end). Due to the engagement with the drive element, the piston rod can thus only move forwards. During dose delivery, the drive element rotates anti-clockwise and the ratchet arms 135 provide the user with small clicks due to the engagement with the ratchet teeth 105, e.g. one click per unit of insulin expelled.

Turning to the dial system, the dose is set and reset by turning the dial member 180. When turning the dial, the reset tube 160, the EOC member 128, the ratchet tube 150 and the scale drum 170 all turn with it due to the dial coupling being in the engaged state. As the ratchet tube is connected to the distal end of the torque spring 155, the spring is loaded. During dose setting, the arm 152 of the ratchet performs a dial click for each unit dialled due to the interaction with the inner teeth structure 142 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 102.

The ratchet 152, 142 between the ratchet tube and the clutch element 140 prevents the spring from turning back the parts. During resetting, the reset tube moves the ratchet arm 152, thereby releasing the ratchet click by click, one click corresponding to one unit IU of insulin in the described embodiment. More specifically, when the dial member is turned clockwise, the reset tube simply rotates the ratchet tube allowing the arm of the ratchet to freely interact with the teeth structures 142 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the push button 190 is pushed in the distal direction by the user as shown in FIG. 3B. The dial coupling 161, 181 disengages and the reset tube 160 decouples from the dial member and subsequently the clutch element 140 disengages the housing splines 104. Now the dial mechanism returns to “zero” together with the drive element 130, this leading to a dose of drug being expelled. It is possible to stop and start a dose at any time by releasing or pushing the push button at any time during drug delivery. A dose of less than 5 IU normally cannot be paused since the rubber piston is compressed very quickly leading to a compression of the rubber piston and subsequently delivery of insulin when the piston returns to the original dimensions.

The EOC feature prevents the user from setting a larger dose than left in the cartridge. The EOC member 128 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 170 is provided with a distal stop surface 174 adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism.

To prevent accidental over-dosage in case something should fail in the dialling mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “overdose” of drug to be expelled, e.g. 3-5 IU of insulin.

The expelling mechanism further comprises an end-of-dose (EOD) click feature providing a distinct feedback at the end of an expelled dose informing the user that the full amount of drug has been expelled. More specifically, the EOD function is made by the interaction between the spring base and the scale drum. When the scale drum returns to zero, a small click-arm 106 on the spring base is forced backwards by the progressing scale drum. Just before “zero” the arm is released and the arm hits a countersunk surface on the scale drum.

The shown mechanism is further provided with a torque limiter in order to protect the mechanism from overload applied by the user via the dial member. This feature is provided by the interface between the dial member and the reset tube which as described above are rotationally locked to each other. More specifically, the dial member is provided with circumferential inner teeth structure 181 engaging a number of corresponding outer teeth 161, the latter being arranged on a flexible carrier portion of the reset tube. The reset tube teeth are designed to transmit a torque of a given specified maximum size, e.g. 150-300 Nmm, above which the flexible carrier portion and the teeth will bend inwards and make the dial member turn without rotating the rest of the dial mechanism. Thus, the mechanism inside the pen cannot be stressed at a higher load than the torque limiter transmits through the teeth.

Having described the working principles of a mechanical drug delivery device, embodiments of the present invention will be described.

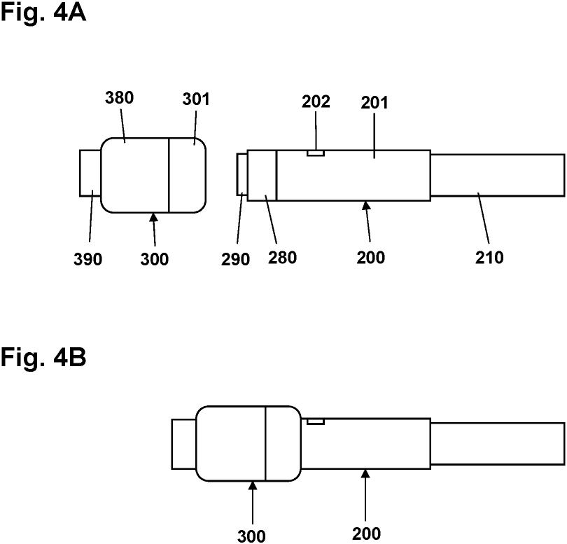

FIGS. 4A and 4B show a schematic representation of a first assembly of a pre-filled pen-formed drug delivery device 200 and a therefor adapted add-on dose logging device 300. The add-on device is adapted to be mounted on the proximal end portion of the pen device housing and is provided with dose setting and dose release means 380 covering the corresponding means on the pen device in a mounted state as shown in FIG. 4B. In the shown embodiment the add-on device comprises a housing portion 301 adapted to be mounted axially and rotationally locked on the drug delivery housing. The add-on device comprises a rotatable dose setting member 380 which during dose setting is directly or indirectly coupled to the pen dose setting member 280 such that rotational movement of the add-on dose setting member in either direction is transferred to the pen dose setting member. The add-on device further comprises a dose release member 390 which can be moved distally to thereby actuate the pen release member 290.

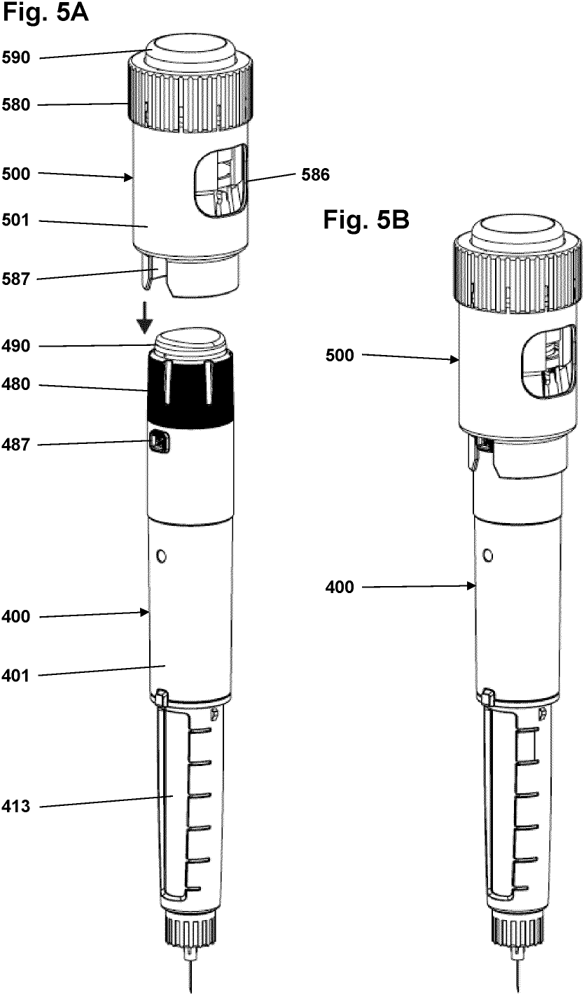

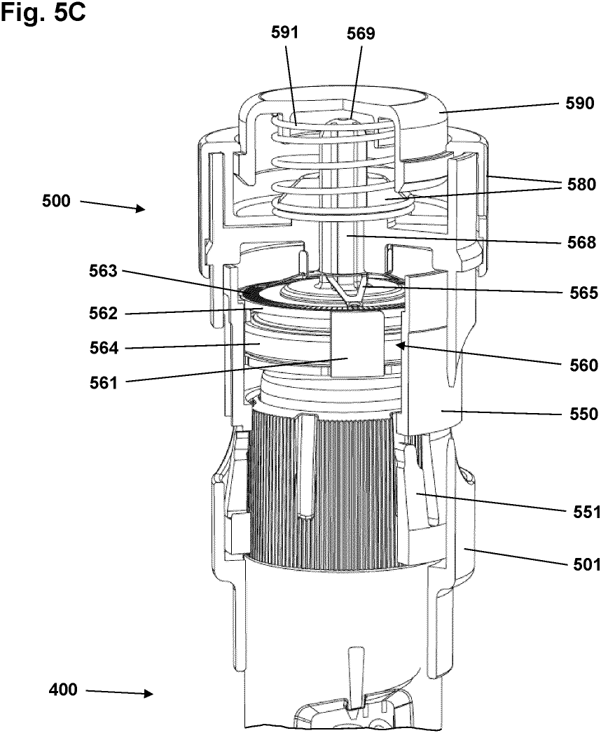

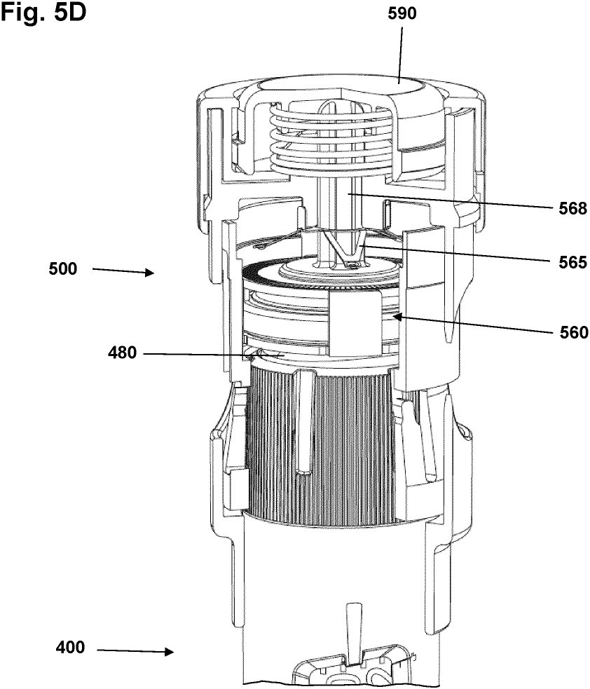

FIGS. 5A and 5B show a more specific embodiment of an add-on dose logging device 500 adapted to be mounted on a pen-formed drug delivery device 400 is shown, the pen device essentially corresponding to a FlexTouch® prefilled drug delivery pen device comprising a housing 401 and a non-removeable drug cartridge 413. The add-on device 500 essentially corresponds to the above-described add-on dose logging device 300 and thus comprises a housing member 501 adapted to be mounted axially and rotationally locked on the drug delivery housing 401 via releasable coupling means. Depending on the length of the housing portion it may be provided with a window allowing the pen scale drum to be observed during dose setting. In the shown embodiment the housing is for illustrative purposes provided with an opening 586 allowing interior components to be seen. The add-on device comprises a rotatable dose setting member 580 which when mounted is coupled to the pen dose setting member 480 such that rotational movement of the add-on dose setting member in either direction is transferred to the pen dose setting member. The add-on device further comprises a dose release member 590 which can be moved distally to thereby actuate the pen release member 490 when mounted.

FIG. 5C shows in a partial cut-away view an add-on dose logging device 500 comprising an outer assembly and a thereto rotationally coupled inner assembly. The outer assembly comprises an outer housing member 501 adapted to be mounted axially and rotationally locked on the drug delivery housing via releasable coupling means 587, 487 (see FIG. 5A), a rotatable dose setting member 580 rotationally free coupled to the proximal end of outer housing member 501, and a dose release member 590 which can be moved distally relative to the housing member and dose setting member to actuate the pen release member 490 (see below). A return spring 591 biases the dose release member to its proximal-most position.

The inner assembly comprises an inner housing member 550 having a proximal portion in which an encoder main assembly 560 is non-rotationally but axially moveable arranged and a distal coupling portion 551 adapted to non-rotationally engage the dose setting member 480. Attached to the encoder main assembly and rotating therewith an actuation rod 568 extends in the proximal direction. The actuation rod is in axially splined engagement with the add-on dose setting member 580 and comprises a proximal end 569 adapted to engage the dose release member 590 as the latter is moved distally. The encoder main assembly comprises a casing 561 in which an encoder PCB 562 and a power source (coin “battery”) 564 is arranged. The encoder PCB comprises a proximal surface on which a circular array of encoder segments 563 is arranged and a distal surface on which processor and transmitter circuitry (not shown) is arranged. The encoder further comprises a stationary (i.e. rotationally locked to the outer housing member) slider member 565 comprising a number of flexible contact arms in sliding engagement with the encoder segments as the encoder main assembly rotates during dose setting. In the shown embodiment the encoder comprises 120 segments and the slider member three contact arms. In a mounted state the encoder main assembly lightly engages the spring-biased pen release member 490, this assuring that the PCB encoder segments are forced into engagement with the flexible arms of the slider.

In a situation of use with the add-on device 500 mounted on the pen device 400 the user sets a dose to be expelled by rotation of the add-on dose setting member 580, this in turn rotating the inner assembly and thus the pen dose setting member. Simultaneously the amount of rotation is measured by the rotary encoder. When the user actuates the add-on dose release member, see FIG. 5D, it is moved distally and engages the actuation rod 568 after which further axial depression of the add-on dose release member is transferred to the pen dose release member 480 via the encoder main assembly 560. During the initial axial movement of the encoder main assembly the encoder slider member 565 disengages the encoder segments allowing a final set dose to be determined by the sensor electronics.

### Power Management

Prior to first dose setting and after each out-dosing, the pen dose setting member (or dial) is left in “zero-position” (0 units). During initial mounting of the add-on device on a (new) pen device it may be necessary to rotate the distal coupling portion into engagement with the pen dose setting member during which operation a dose will be “set”. Correspondingly, the user may zero-adjust the sensor assembly by e.g. three pushes on the add-on release member. This operation may also be used during subsequent use of the device to reset the sensor. Due to mechanical slack in the components, the measurements of this position may have some inaccuracy and will not necessarily give identical positions after each out-dosing. However, the position of the dial after the most recent out-dosing will be stored, as well as an initial first measurement as the sensor electronics (or “system”) is turned on for the first time.

The system may then go into a low power consumption sleep-mode and partially wake-up and measure the position of the dial with short time intervals of once a second or so. If no change is detected, the system returns to sleep for an additional time interval and again checks if position has changed since last. A small switch may also be implemented, such that any dial-up rotation will trigger the switch and turn the system on.

If a switch is provided and triggered, or if the system wakes up partially after a time interval and detects a change of position, the system switches to fully awake mode and measures the position with very short time intervals, e.g. 10 or 100 times per second. If no change is detected for a minute or two, the systems goes back to sleep-mode or power off, if fitted with a switch.

If a change in position is registered, the type of change may be identified based on the measurements, e.g. small jittering due to vibrations of the device and slack in the mechanism, whereby positions may change slightly. The system may then establish a range of positions within which the system should ignore the changes and return to sleep mode.

If the measurements appear to indicate the beginning of a dial-up (rotational positions measured increasing in dial-up direction and exceeding 0,5-1 unit, the system switch into dial-mode and handle measured data according the algorithm described in the following. In the shown embodiment the sensor assembly is designed to essentially count the number of segments swiped by the slider member during dose setting, however, for a given number of segments a given rotational position can be determined, this allowing the counter to “catch up” after a wake-up event as well as identifying when the direction of rotation changes during dose setting.

### Signal Processing

The present invention incorporates domain knowledge based Algorithmic Auto Calibration (referred to as “AAC” in the following) used for the signal processing of the encoder measurements.

### 1. Introduction

AAC uses the data from dial and not from dosing out. Regulations limits the allowable deviance between dialed and actually out-dosed volume. Since actually out-dosed volume is not measured in regular injection devices, but assumed to be the same (within regulatory requirements) as the dialed size of dose, it is assumed that the user injects what is dialed for, and the injected dose is the actually dialed dose. The overall objective is to estimate the injected dose from the erroneous measurements, using the domain knowledge and the information extracted from the dial data.

If d is the intended (actual) dialed dose, dₘ is the dialed dose measured by the encoder and e is the measurement error, the dialed dose measured, dₘ, will be given by:

$${d}_{m} = d + e \qquad \text{(1)}$$

The physical unit of the quantities in (1) is rotational degrees. Depending on the actual rotary sensor implemented the resolution may be different from 1 degree.

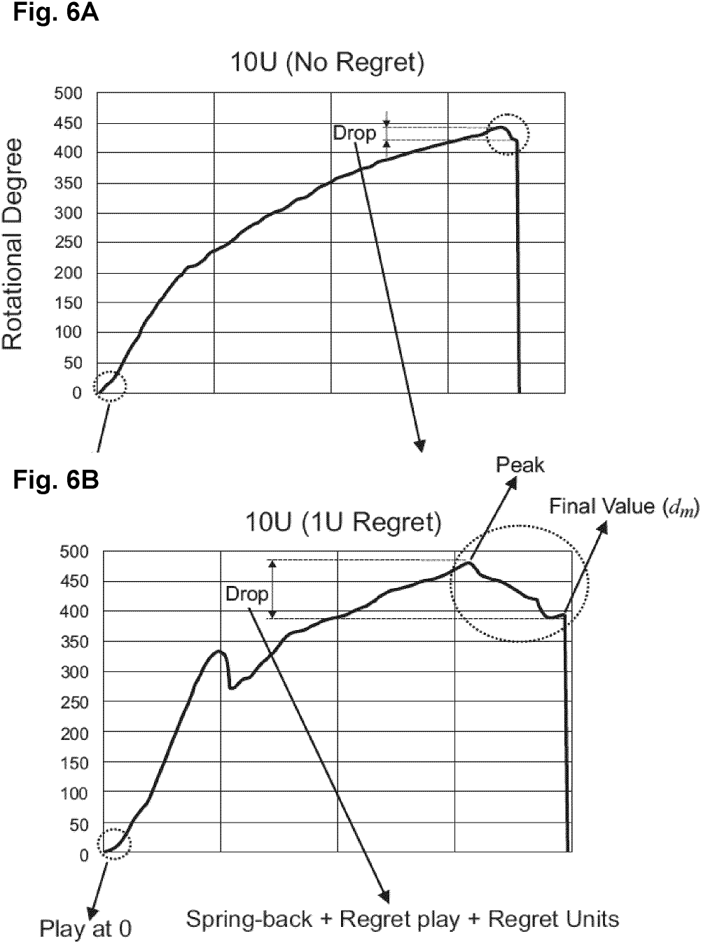

It is hypothesized that the error has two components: (i) tolerance at zero and (ii) tolerance at regret. A dial with regret means that the user dials to a given number of units, then regrets and dials back to a smaller number of units and finally doses out. FIG. 6 illustrates the encoder data for dialing in two cases with ‘regret’ respectively ‘no regret’.

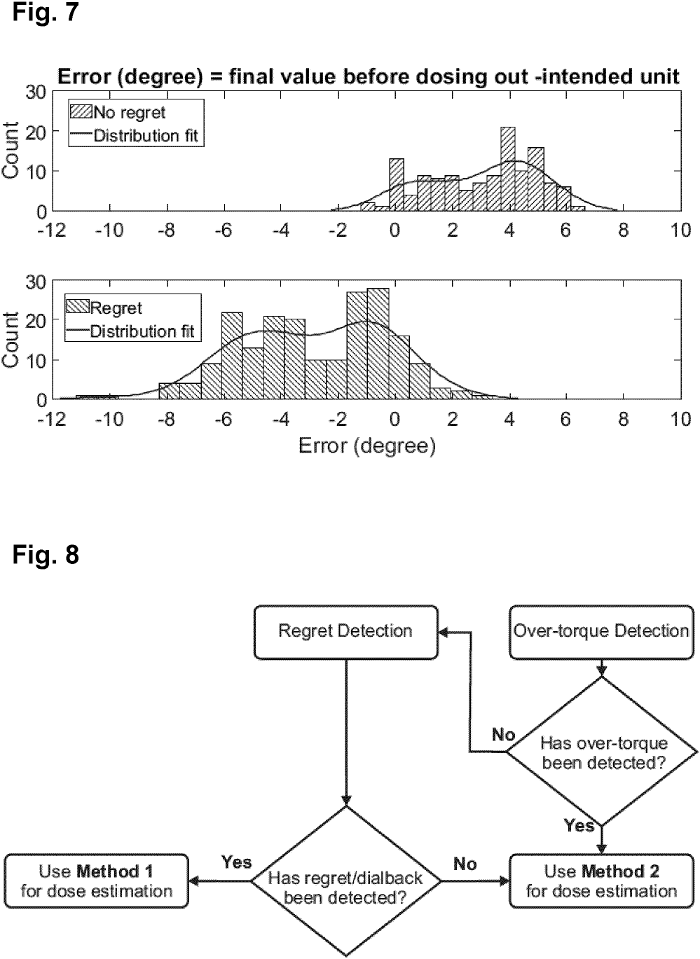

FIG. 7 shows distributions of the actual error (e) from the measured dialed doses in cases of ‘regret’ and ‘no regret’. Comparing the two distributions indicates that the dials without regret have a positively biased error while the dials with regret have a negatively- biased error. Error (degree) equals final value before out-dosing minus intended number of units.

Before turning to the individual components in an exemplary embodiment of the present invention, FIG. 8 shows a high-level description of an embodiment of AAC. The optional incorporation of over-torque detection depends on whether it is relevant for a given pen design or, if it is, whether it is implemented.

### 2. Over-Torque Detection

A drug delivery pen device adapted to expel a desired user-set dose amount of drug will allow a given maximum of units to be set and subsequently expelled. When the maximum set dose has been reached, e.g. 60 or 80 units, or an end-of-content stop is encountered, the setting mechanism may be provided with a “hard stop” preventing any further rotation of the dose dial to increase the dose size. Alternatively, to protect the dose setting mechanism, an over-torque protection may be incorporated in the pen design allowing the user to rotate the dial without increasing the set dose, e.g. as disclosed in WO 2018/041899.

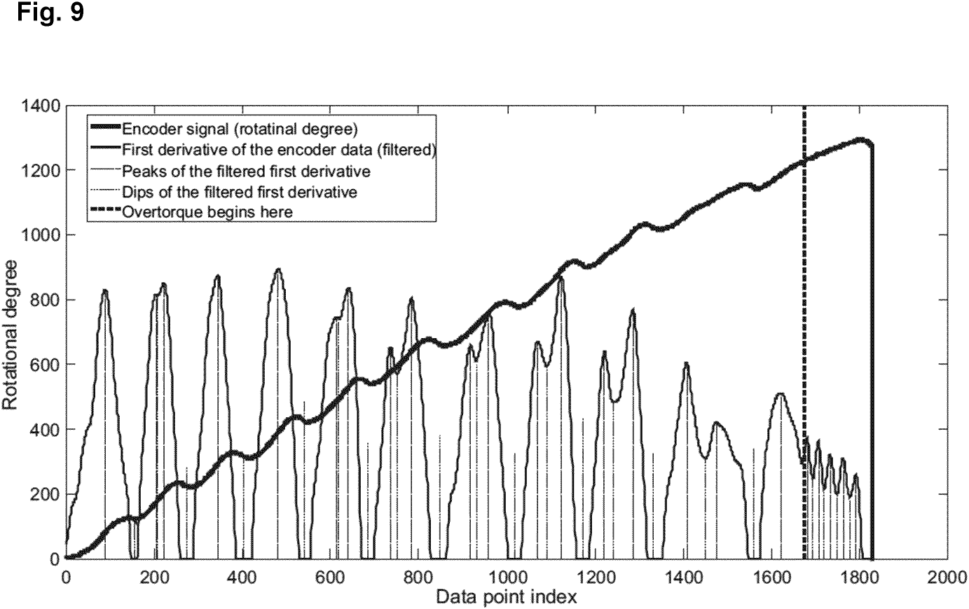

An over-torque condition then occurs when the user reaches the maximum dose or the end-of-content of the drug cartridge, while he or she continues dialing. In this case the encoder continues measuring the increase in the rotational degree due to rotation of the dial, but the dialed dose size as shown in the scale drum window does not change. FIG. 9 illustrates the encoder data when the user dials and starts to over-torque at t_{overtorque}. The over-torque detection algorithm has the following steps:
1. Filtering the data: This is a moving average smoothing filter that mitigates noise in the encoder signal.
2. Taking the first derivative of the filtered signal.
3. Filtering the first derivative: this is a zero-phase filtering which does not introduce extra filtering delay into the signal. To perform a zero-phase filtering, after filtering the data in the forward direction, we reverse the filtered sequence and run it back through the filter. We reverse the filtered signal again, which gives the zero-order filtered signal.
4. Detecting the local peaks (maximum) and dips (minimum) of the first derivative: this is a peak/ dip detection algorithm.
5. Comparing the area between the consecutive peaks and dips with a threshold. If the area is less than the threshold, it is an over-torque.

FIG. 9 also shows the peaks and dips indicators extracted for over-torque detection. The over-torque indicator is a flag, which is activated when the over-torque is detected. When over-torque is detected, the last value just before the over-torque indicator is considered as dₘ for dose estimation.

### 3. Regret Detection

In the beginning the algorithm detects if the user dialed the units with regret. Using an encoder with e.g. one degree of resolution, each insulin unit on the scale drum occupies 15 degrees in the encoder. The regretted insulin units in FIG. 6B is estimated as:

$$r = \left⌊\frac{d r o p - S p r \hat{i n g B} a c k}{15}\right⌋ , \qquad \text{(4)}$$

where drop = peak - final value and └ ┘ indicates the integer part of the division result. If r is larger than zero, dial has regret.

$$S p r \hat{i n g B} a c k$$

is an estimate of the spring back movement occurring just after the peak dialed degree and its computation is described below.

It should be noted that regret detection in more complex dialing patterns uses the last event before out-dosing. If the last event is a regret, the AAC uses method 1 in FIG. 8 and if the last event is a dial up (no regret) it uses method 2.

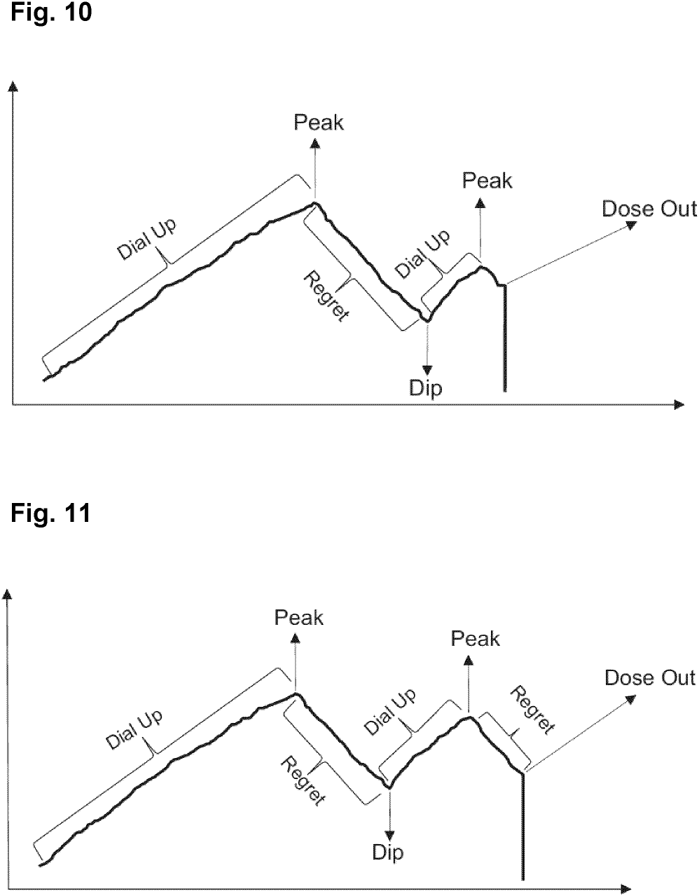

In general, an event of dialing consists of an arbitrary sequence of events depending on the user’s actions, e.g., the sequence of events in FIG. 10 is: dial up, regret, dial up, dose out and the sequence of events in FIG. 11 is: dial up, regret, dial up, regret, dose out.

AAC uses a peak and dip detection algorithm to find the last peak in the dial sequence and uses it to detect whether the dial had regret. The algorithm uses a systematic approach to differentiate the major peaks and dips from the minor spurious peaks and dips caused by the noise in the signal.

### 4. Method 1: Estimating the Actual Dose When Dial Has Regret

The essence of this method is estimating the intended insulin dose by estimating the error caused by regretting which is called ‘regret play’. Although the regret play is limited to the range of (0-15) degrees, it can have a different value in each dial event. The aim is to estimate the regret play for every dial and correct for it accordingly.

The mechanical interpretation of regret play can be explained as follows. When the user regrets and then out-doses, the drop (excluding the regretted units and spring back) can be computed as:

$$\begin{matrix}d r o p = p e a k - f i n a l v a l u e b e f o r e d o s i n g o u t - s p r i n g b a c k - \\ r e g r e t u n i t s \times 15 ,\end{matrix}$$

which is larger than the drop when there is no regret, i.e.,

$$d r o p = p e a k - f i n a l v a l u e b e f o r e d o s i n g o u t - s p r i n g b a c k .$$

The mechanism behind this is that in case of regret the drive spring (in this case) generates a torque in the opposite direction of dialing up. The spring tends to regret and jump slightly more than the expected degree of regret×15 due to the spring overload. This extra backward jump of the spring is called ‘regret play’ and the AAC computes it as:

$$R e g \hat{r e t P} l a {y}_{i} = \left(d r o {p}_{i} - S p r i \hat{n g B} a c k\right) mod 15 \qquad \text{(5)}$$

Where mod is the remainder of the division,

$$( d r o {p}_{i} - S p r i \hat{n g B} a c k ) / 15 ,$$

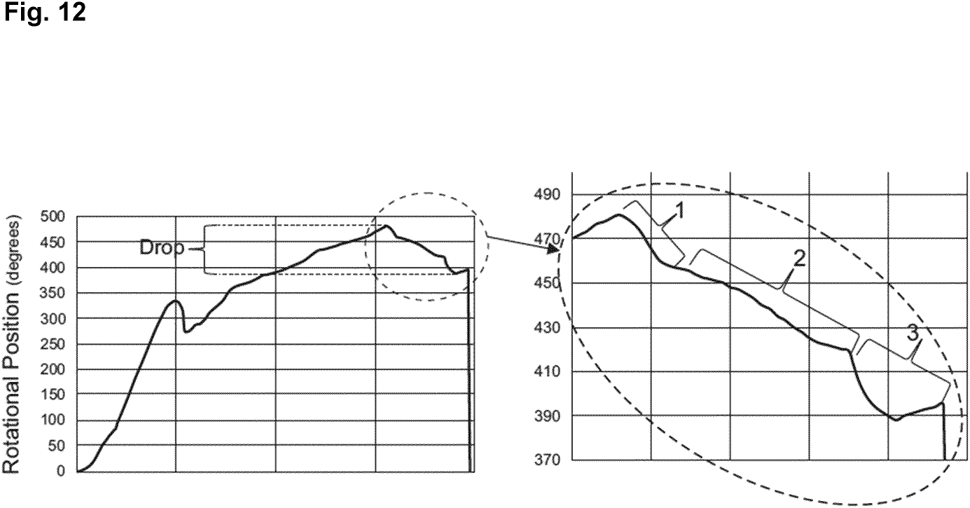

for the dial event i. FIG. 12 depicts the contribution of spring back, regret units and regret play in the signal drop.

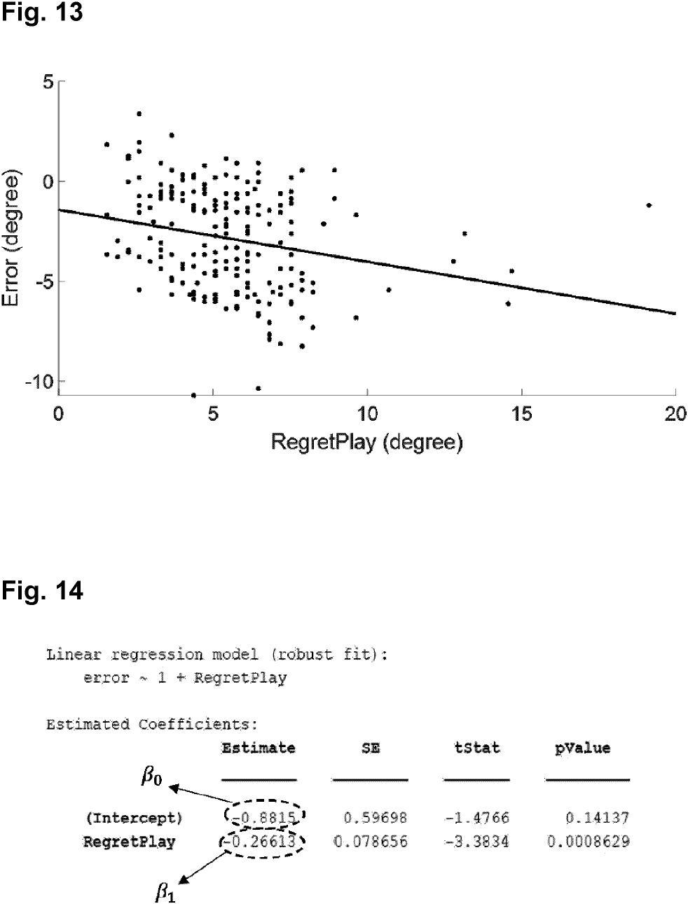

In respect of error prediction FIG. 13 shows the scatter plot of the actual error versus the estimated regret play extracted according to (5). The actual error is:

$$e = f i n a l v a l u e b e f o r e d o s i n g o u t - d ,$$

where d is calculated using the conversion in (3).

FIG. 13 suggests that there is a correlation between the error and regret play. Correspondingly, the null hypothesis that there is not a linear trend between the error and regret play was tested by performing the linear regression,

$$e = {β}_{0} + {β}_{1} \times Re \hat{g r e t P} l a {y}_{i} ,$$

and checking if the null hypotheses β₀ = 0 and β₁ = 0 can be rejected.

By fitting the linear model to the data from 200 performed experimental dosing events with regret, the linear regression rejected the null hypotheses of β₁ with P-value < 0.05, however it did not reject β₀ = 0. FIG. 14 shows estimated coefficients of β₀ and β₁.

The presence of a linear relation between regret play and error indicates that the estimated regret play can estimate the measurement error using the linear model:

$${\hat{e}}_{i} = {β}_{1} \times R e g \hat{r e t P} l a {y}_{i} , \qquad \text{(6)}$$

where êᵢ is the estimated measurement error for dial event i.

Using the data from the training set, we realized that adding an additional term βₒᵤₜₗᵢₑᵣ gives a better estimation of outlier errors. Using this prediction model, method 1 for estimating the actual dialed dose is described as:

$${\hat{e}}_{i} = {β}_{o u t l i e r} + {β}_{1} \times R e g \hat{r e t P} l a {y}_{i} , \qquad \text{(7)}$$

$${\hat{d}}_{i} = {d}_{m , i} - {\hat{e}}_{i} \qquad \text{(8)}$$

êᵢ is the estimated measurement error, β₁ is the regression coefficient, βₒᵤₜₗᵢₑᵣ is the outlier coefficient, d̂ᵢ is the estimated intended dose (in degree) for event i and d_{m,}ᵢ is the measured dose for event i. β₁ and βₒᵤₜₗᵢₑᵣ are estimated offline using a training dataset, while

$$R e \hat{g r e t P} l a {y}_{i}$$

is estimated online for event i. The estimated units of insulin in computed as

$$\hat{u} = \left[\frac{\left({\hat{d}}_{i} - 10\right)}{15} + 1\right] \qquad \text{(9)}$$

Estimating the spring back factor: according to (5) and (7) prediction of error requires an estimate of spring back factor. The spring back factor is the fall in the rotational degree due to the backward movement of the Torque Spring when the user releases the dial. AAC estimates the spring back from the dialed events without regret. For these dial events, the drop from the peak to final value before dosing out is equivalent to the spring back factor FIG. 6A.

Using a train dataset,

$$S p r \hat{i n g B} a c k$$

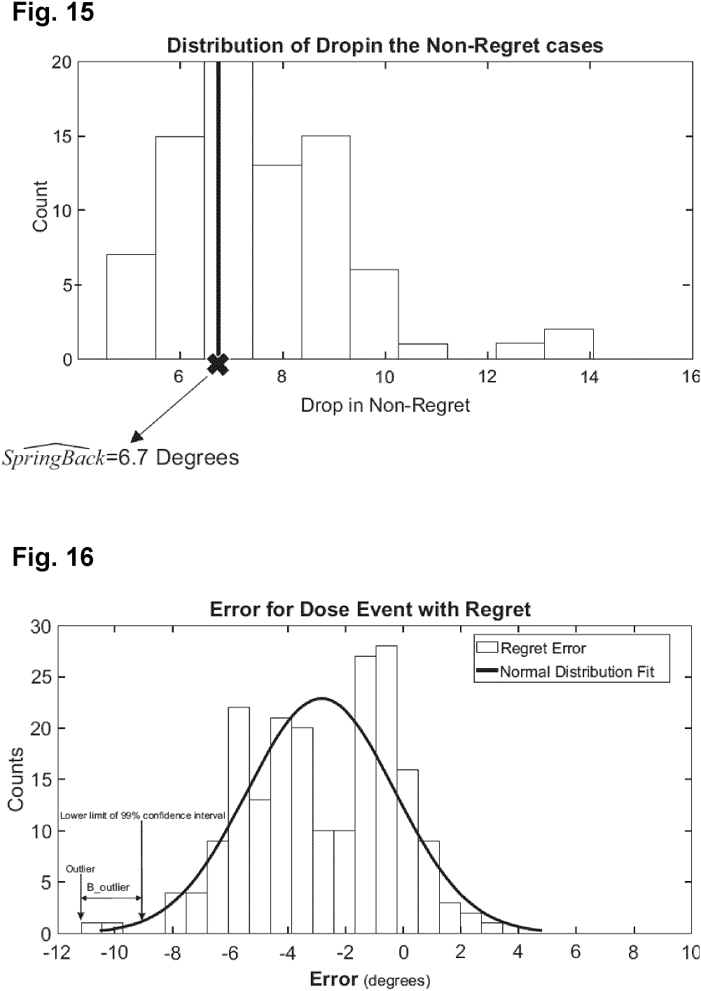

is estimated as a population parameter and is defined as (see FIG. 15):

$$\begin{matrix}S p r \hat{i n g B} a c k 15 \% p e r c e n t i l e o f d i s t r i b . o f t h e ′ n o n r e g r e t D r o p ′ i n \\ t h e t r a i n d a t a s e t\end{matrix} \qquad \text{(10)}$$

Estimating βₒᵤₜₗᵢₑᵣ for the regression: The outlier is the error, which is outside the 99% confidence interval of the normal distribution fit. The linear regression assumes a normal distribution for the error.

If the error distribution deviates from normality with large outliers, the estimated error by

$$\hat{e} = {β}_{1} \times R e \hat{g r e t P} l a {y}_{i}$$

is not a good estimate of the outlier errors.

Based on performed experiments, the outlier regret errors in the left side of the distribution cause bias in the estimate of the insulin unit. FIG. 16 shows the distribution of error and the position of the outliers in dials with regret.

The AAC shifts the outlier regret errors into normal distribution by adding the coefficient, βₒᵤₜₗᵢₑᵣ, to the estimated error as in (7).

βₒᵤₜₗᵢₑᵣ is the minimum outlier correction without distorting the non-outlier error values. AAC uses the train dataset to estimate βₒᵤₜₗᵢₑᵣ as:

$$\begin{matrix}{β}_{o u t l i e r} = \text{minimum error in regret dataset} - \text{lower limit of 99\% Cl} \\ \text{of error in regret dataset}\end{matrix}$$

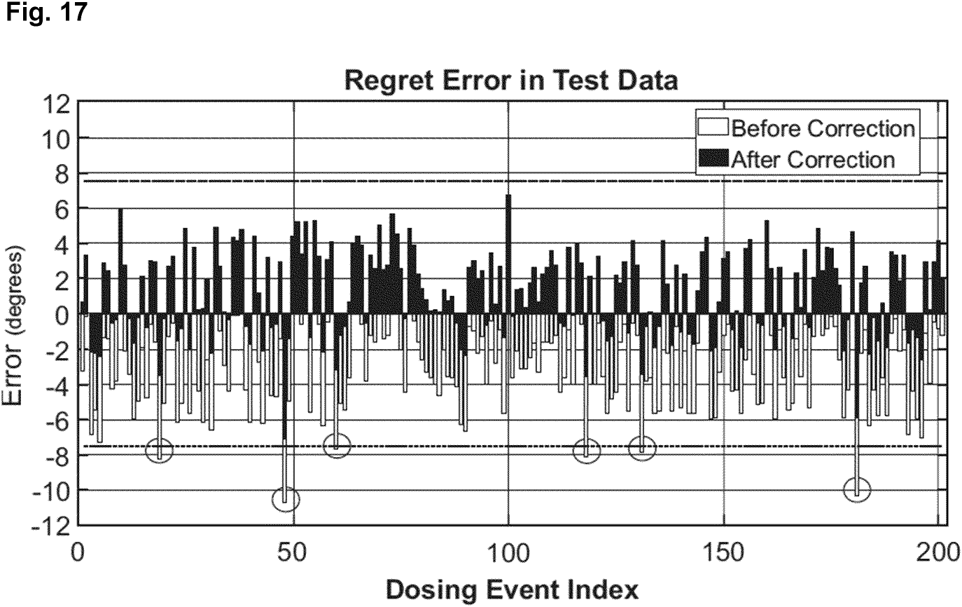

FIG. 17 presents the results of applying AAC on the dials with regret. The AAC decreased the error and estimated the intended actual insulin unit with 100% accuracy in the test dataset. The measurement error in the test dataset, which is partially mitigated after applying AAC method 1 on the dial data with regret. The error for each dial event before AAC correction is computed as eᵢ = d_{m,}ᵢ - dᵢ and after AAC correction it is computed as: eᵢ = d̂ᵢ - dᵢ, where d_{m,}ᵢ is the final value before dosing out, dᵢ is the actual intended dose and d̂ᵢ is the estimated intended dose as in (8). dᵢ, d̂ᵢ and d_{m,}ᵢ are rotational degrees.

### 5. Method 2: Estimating the Actual Dose When Dial Does Not Have Regret

When user does not regret while dialing and directly dials up before dosing out, the most important part of the error is the error at zero position, namely ‘zero play’. The term zero play means that when the scale drum is at zero, the rotational degree measured by the encoder is not zero.

Zero play is in fact a bias in the measurement and can change in the range [-7, 7]. We describe how the AAC compensates for the zero-play bias in two cases: dialing up without any middle rest and dialing up with at least one middle rest.

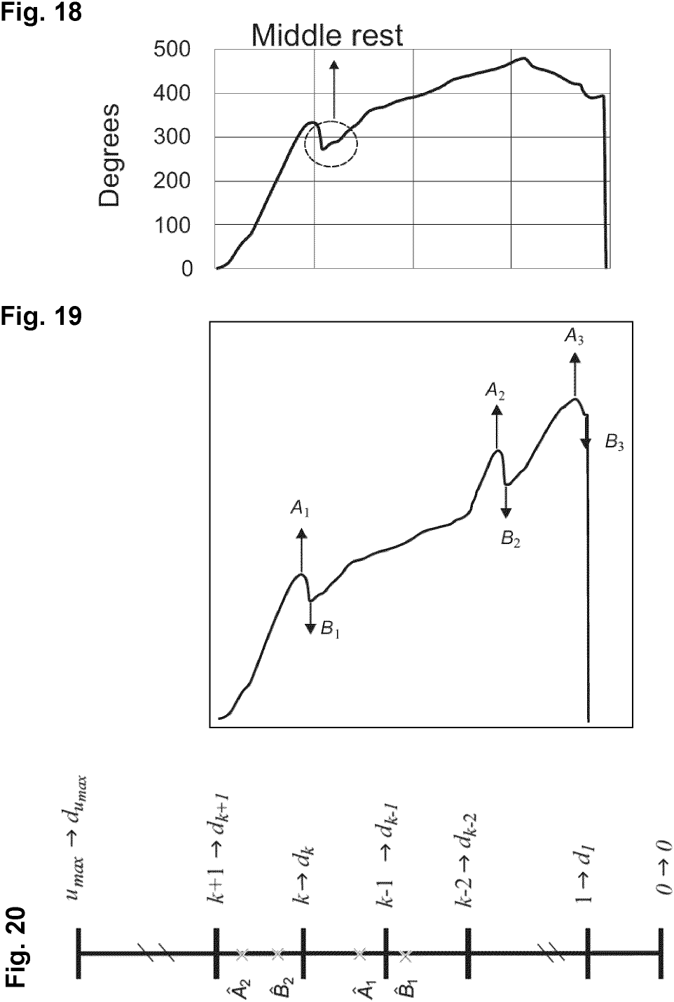

A dial without a middle rest is when the user directly dials up to the intended dose without resting his hand. A middle rest occurs when the user releases his hand from the Dial on the way to dial up and continues to dial up after a short pause. This creases a notch in the encoder signal as indicated in FIG. 18.

In the case of dialing up without middle rest, the zero-play bias is estimated as the mean of the error distribution of the train dataset without regret. i.e.,

$${\hat{e}}_{0} = m e a n \left({\left\{{d}_{m , l} - {d}_{l}\right\}}_{l = 1 , ⋯ , N}\right) , \qquad \text{(12)}$$

where N is the number of train dosing events.

The estimated actual dose is computed as:

$${\hat{d}}_{i} = {d}_{m , i} - {\hat{e}}_{0} , \qquad \text{(13)}$$

with (9) estimating the intended insulin unit.

When the user has at least one middle rest in the dial, the information in rest positions can give a more precise estimation of the zero play and as a result a more precise estimation of the intended insulin unit. Let us assume that the dial has n rests including the final rest which occurs at the final value before dosing out, e.g., FIG. 19 shows a dial event with two middle rests (B₁ and B₂) plus the final rest (final value) at B₃, i.e. three rests in total.

Each rest at position j gives an estimate of zero play according to:

$$\begin{matrix}{u}_{r e s t , j} = \left[\frac{\left({B}_{j} - 10\right)}{15} + 1\right] , {\hat{e}}_{0 , j} = {B}_{j} - \left(\left({u}_{r e s t , j} - 1\right) \times 15 + 10\right) , \text{for} j = \\ 1 , ⋯ , n ,\end{matrix} \qquad \text{(14)}$$

where n is the total number of rests.

The overall zero play estimate is:

$${\hat{e}}_{0} = m e a n \left({\left\{{\hat{e}}_{0 , j}\right\}}_{j = 1 , ⋯ , n}\right) . \qquad \text{(15)}$$

The AAC corrects for the zero play bias by estimating new rest positions as:

$${\hat{A}}_{j} = {A}_{j} - {\hat{e}}_{0} \text{and} {\hat{B}}_{j} = {B}_{j} - {\hat{e}}_{0} , \text{for} j = 1 , ⋯ , n . \qquad \text{(16)}$$

From the mechanics of the ratchet, the torque spring, the scale drum, the piston rod and the reset tube, it is known that at each rest position the difference between Aⱼ and Bⱼ is the spring back factor and therefore Aⱼ and Bⱼ must be within the same click of insulin unit in the Scale Drum. This mechanical characteristic can be called a ‘within the same click’ property.

̂The correction for zero play should preserve this property, i.e., Âⱼ and Bj should be within the same click of insulin unit, otherwise the estimation of ê₀ is not valid and it should be adjusted. This is illustrated graphically in FIG. 20 showing the position of the rest points with respect to the unit markers on the scale drum. For every rest, the legitimate positions for Âⱼ and B̂j are within the same ‘interval’. The rest position R₁ = [B̂₁,Â_{1]}^{T} is not legitimate, while R₂ = [̂B₂, Â_{2 ]}^{T} is legitimate.

Let D be the vector of unit markers, D = [d₁,···, and rest j be defined as Rⱼ = [B̂ⱼ, Âⱼ]^{T} for j = 1, ···, n, where Âⱼ and B̂j are from (16). For every rest position, let D_{Aj} = D - Âⱼ and D_{Bj} = D - B̂ⱼ, and S_{Aj} = sgn(D_{Aj}) and S_{Bj} = sgn(D_{Bj}). The sign function, sgn, is defined as:

$$s g n \left(x\right) = \left\{\begin{matrix}- 1 i f x < 0 , \\ 0 i f x = 0 , \\ 1 i f x > 0.\end{matrix}\right\}$$

S_{Aj} and S_{Bj} are the vectors of -1, and 1. Due to the known mechanical properties, the occurrence of 0 in S_{Aj} and S_{Aj} is not probable.

The AAC checks for the ‘within the same click’ property of the rest positions, Rⱼ = [B̂ⱼ, Âⱼ]^{T}, and corrects the estimate of the intended dose accordingly. The pseudocode is described as follows.

While the relation S_{Bj} = S_{Aj} is not satisfied for all j = 1,···, n, Do
1. D_{Aj} = D - Âⱼ = [Δ_{Aj,1}, Δ_{Aj,2},⋯,Δ_{Aj,u}ₘₐₓ]^{T} and D_{B}ⱼ = D - B̅ⱼ = [Δ_{Bj,1}, Δ_{Bj,2},⋯Δ_{Bj,u}ₘₐₓ]^{T}.
2. S_{Aj} = sgn(D_{Aj}) and S_{Bj} = sgn(D_{Bj}),
3. Is S_{Bj} = S_{Aj} satisfied for all j = 1, ...,n?
Yes: zero play correction is complete. The estimated actual dose (degree) is B̂ₙ and the estimated insulin unit is
$$\hat{u} = \left[\frac{\left(\hat{{B}_{n}} - 10\right)}{15} + 1\right]$$
No: there is at least one rest position that S_{Bj} ≠ S_{Aj}. To correct this, perform the following recursive procedure:
   For each rest position that S_{Bj} ≠ S_{Aj}, find |Δeⱼ| = min {|∈_{A}|, |∈_{B}|} in D_{Aj} = D_{Aj} + ∈_{A} and D_{Bj} = D_{Bj} + ∈_{B} that makes S_{Bj} = Sₐⱼ. For example, assume that D_{Aj} = [-50, -35, -20, -5, 10,...,, and D_{Bj} = [-38,-23,-8, 7, 22, ⋯ ]^{T} which results in S_{Aj} = [-1, -1, -1, -1, 1, ⋯ ]^{T}and S_{Bj} = [-1, -1, -1, 1, 1, ⋯ ]^{T}. Comparing S_{Aj} and S_{Bj} indicates that Âⱼ is located after d₄, i.e., Âⱼ > d₄ and B̂ⱼ is located before d₄, i.e., B̂ⱼ < d₄. To satisfy S_{Bj} = S_{Aj}, either -5 in D_{Aj} should be shifter up to become positive, i.e., -5+∈_{A} > 0 which gives ∈_{A} = 6 (with an encoder resolution of 1 degree), or 7 in D_{Bj} should be shifter down to become negative, i.e., 7+∈_{B} < 0 which gives ∈_{B} = -8. Therefore |Δeⱼ| = min{|∈_{A}|, |∈ₐ|} = 6 and Δeⱼ = 6.
   Compute Δe = min{Δeⱼ }, where Δeⱼ values are computed in the previous step.
   Set Âⱼ = Âⱼ -Δe and B = B̂ⱼ -Δe.
   Go to step 1.

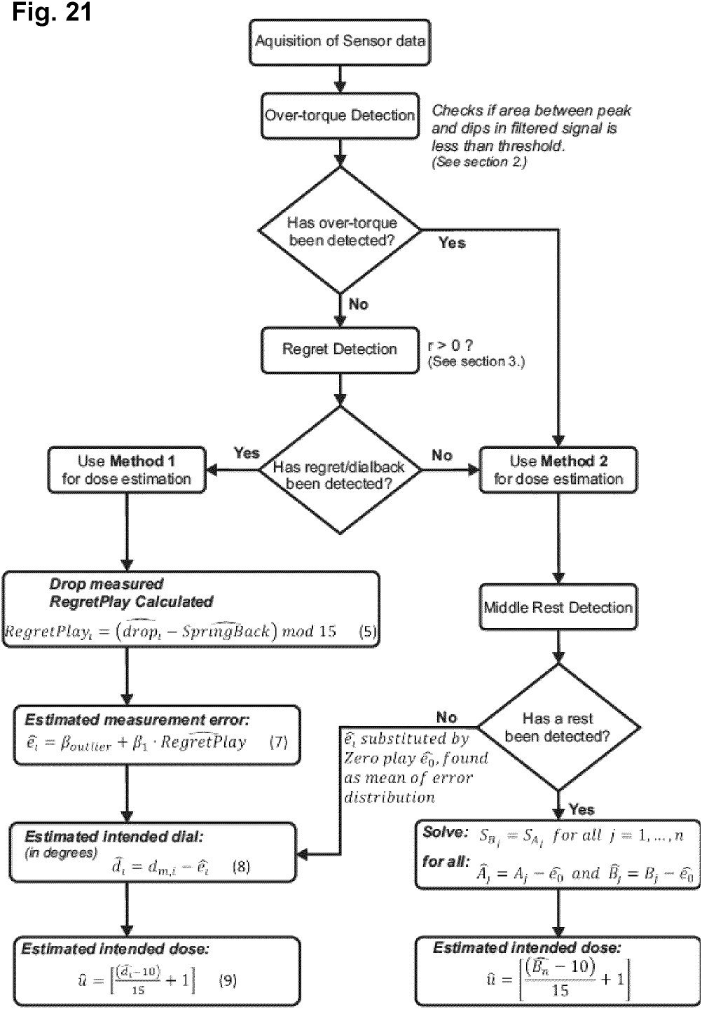

In summary, FIG. 21 shows an overview of the above-described embodiment of the AAC signal processing of the encoder signals from a rotational encoder in an add-on unit for an injection device.

In the above devices and methods providing dose size measurements for a dose setting mechanism has been described which may be implemented to create a dose log for a given drug delivery device based on the dose set by the user, this in contrast to an arrangement in which the size of an expelled dose is measured.

Indeed, such a measured set dose will only correspond to a subsequently expelled (and injected) dose when the drug delivery device is used as intended and corresponding to the prescribed way of use, e.g. setting a dose to be expelled, inserting an injection needle subcutaneously and subsequently actuate the drug delivery device to fully expel the set dose. Although this may be the recommended way of use, other use scenarios can be envisioned and may be relevant.

For example, for larger dose amounts to be injected, it is common to split a given set dose into e.g. two injections. Such a situation of use could be addressed by detecting the amount of time between two injections and the length of a given injection, the necessary time stamps being provided by the encoder electronics as the encoder slider disengage and reengage. For example, for a set dose of 80 units a first out-dosing event of 15 seconds, a pause of 30 seconds and a second out-dosing event of 15 seconds could be interpreted as the full dose of 80 units having been expelled/injected. To address the issue of a fully or partly regretted set dose, the encoder electronics may be adapted to measure a “negative” dose, i.e. a not-expelled set dose being reset to zero.

To further assure that measured dose sizes are correctly saved to a dose log, each dose event may require the user to accept (or correct) a given dose entry. This could typically take place on the device, e.g. smartphone, receiving the dose event data.

Alternatively, the measured set dose could also be used in combination with a measured expelled dose, the two measured values allowing a check of the measurements to be performed and allow potential malfunctions to be detected and addressed.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery system, comprising:
a housing defining a reference axis for rotation,
a drug reservoir or structure for receiving a drug reservoir,
drug expelling structure comprising:
a dose setting member adapted to (i) incrementally rotate in a first direction to set a dose, and (ii) incrementally rotate in an opposed second direction to reduce a set dose, the dose setting member having a rotational slack in each incremental rotational position, and
an actuation member adapted to cause a final set dose amount to be expelled,
a rotary sensor adapted to detect the amount of rotation of the dose setting member relative to the housing during dose setting,
a memory, and
processor structure adapted to:
detect a first dose setting pattern when the final dose was set by rotating the setting member in the first direction,
detect a second dose setting pattern when the final dose was set by rotating the setting member in the second direction,
when a first dose setting pattern is detected: based on the detected amount of rotation calculating a corrected amount of rotation using a first algorithm compensating for a slack-induced error generated corresponding to the first dose setting pattern,
when a second dose setting pattern is detected: based on the detected amount of rotation calculating a corrected amount of rotation using a second algorithm compensating for a slack-induced error generated corresponding to the second dose setting pattern, and
store in the memory calculated corrected amounts of rotation or dose data corresponding thereto.

2. A drug delivery system as in claim 1, the drug expelling structure further comprising:
a drive spring for expelling a set amount of drug from the drug reservoir,
the dose setting member being adapted to (i) incrementally rotate in a first direction to set a dose and strain the drive spring correspondingly, and (ii) incrementally rotate in an opposed second direction to reduce a set dose and unstrain the drive spring correspondingly,
wherein the actuation member is a release member adapted to release the strained drive spring to expel a final set dose amount.

3. A drug delivery system as in claim 2, the system being in the form of an assembly comprising a drug delivery device and an add-on device adapted to be releasably mounted on the drug delivery device, wherein the drug delivery device comprises:
the housing,
the drug reservoir or the structure for receiving a drug reservoir, and
the drug expelling structure comprising a drug delivery device dose setting member and a drug delivery device release member,
the add-on device comprising:
the rotary sensor, and
the processor structure.

4. A drug delivery system as in claim 3, wherein the add-on device further comprises:
an add-on housing being releasably attachable to the drug delivery device housing,
an add-on dose setting member, and
an add-on release member axially moveable relative to the add-on dose setting member between a dose setting state and a dose expelling state,
wherein:
the add-on dose setting member is adapted to non-rotationally engage the drug delivery device dose setting member,
the rotary sensor is adapted to detect the amount of rotation of the add-on dose setting member relative to the add-on housing during dose setting, and
when in a mounted state, the add-on release member is adapted to release the drug delivery device release member when moved from the dose setting state to the dose expelling state.

5. A drug delivery system as in claim 1, wherein the rotary sensor is de-activated when the add-on release member is moved from the dose setting state to the dose expelling state.

6. A drug delivery system as in claim 1, the system being in the form of an assembly comprising a drug delivery device and an add-on device adapted to be releasably mounted on the drug delivery device, wherein the drug delivery device comprises:
the housing,
the drug reservoir or the structure for receiving a drug reservoir, and
the drug expelling structure comprising a drug delivery device dose setting member and a drug delivery device actuation member,
the add-on device comprising:
the rotary sensor, and
the processor structure.

7. A drug delivery system as in claim 1, the drug expelling structure further comprising:
a piston rod adapted to engage and axially displace a piston in a loaded cartridge in a distal direction to thereby expel a dose of drug from the cartridge, and
a drive member coupled directly or indirectly to the piston rod,
wherein, when the drug expelling structure comprises a drive spring:
the drive spring is coupled to the drive member, and
the release member is adapted to release the strained drive spring to rotate the drive member to expel the set dose amount.

8. A drug delivery system as in claim 1, wherein the processor structure for a detected first dose pattern is adapted to:
detect a dose setting pause between two consecutive dose setting rotations in the first direction, and
when one or more dose setting pauses are detected: based on the detected amount of rotation calculating a corrected amount of rotation using a third algorithm compensating for a slack-induced error generated corresponding to the first dose setting pattern when one or more dose setting pauses have been detected.

9. A drug delivery system as in claim 1, wherein:
the drug expelling structure comprises an over-torque mechanism allowing the dose setting member to be rotated further in the first direction when a predetermined maximum dose has been set, and
the processor structure is adapted to detect an over-torque condition and calculate the amount of rotation of the dose setting member relative to the housing corresponding to the set maximum dose.

10. A drug delivery system as in claim 1, further comprising:
transmitter structure adapted to transmit dose related data to an external receiver.

11. An add-on device adapted to be releasably mounted on a drug delivery device, the drug delivery device comprising:
a device housing defining a reference axis for rotation,
a drug reservoir or structure for receiving a drug reservoir, and
drug expelling structure comprising:
a device dose setting member adapted to (i) incrementally rotate in a first direction to set a dose, and (ii) incrementally rotate in an opposed second direction to reduce a set dose, the dose setting member having a rotational slack in each incremental rotational position, and
a device actuation member adapted to cause a final set dose amount to be expelled,
the add-on device comprising:
an add-on housing being releasably attachable to the device housing,
an add-on dose setting member adapted to non-rotationally engage the device dose setting member,
an add-on actuation member axially moveable relative to the add-on dose setting member between a dose setting state and a dose expelling state, the add-on actuation member being adapted to, when in a mounted state, actuate the device actuation member when moved from the dose setting state to the dose expelling state,
a rotary sensor adapted to detect the amount of rotation of the add-on dose setting member relative to the add-on housing during dose setting, and
processor structure adapted to:
detect a first dose setting pattern when the final dose was set by rotating the add-on dose setting member in the first direction,
detect a second dose setting pattern when the final dose was set by rotating the add-on dose setting member in the second direction,
wherein:
when a first dose setting pattern is detected: based on the detected amount of rotation calculating a corrected amount of rotation using a first algorithm compensating for a slack-induced error generated corresponding to the first dose setting pattern, and
when a second dose setting pattern is detected: based on the detected amount of rotation calculating a corrected amount of rotation using a second algorithm compensating for a slack-induced error generated corresponding to the second dose setting pattern,
wherein:
the add-on dose setting member is adapted to non-rotationally engage the dose setting member,
the rotary sensor is adapted to detect the amount of rotation of the add-on dose setting member relative to the add-on housing during dose setting, and
when in a mounted state, the add-on actuation member is adapted to actuate the device actuation member when moved from the dose setting state to the dose expelling state.

12. An add-on device as in claim 11, the drug expelling structure further comprising:
a drive spring for expelling a set amount of drug from the drug reservoir,
the dose setting member being adapted to: (i) incrementally rotate in a first direction to set a dose and strain the drive spring correspondingly, and (ii) incrementally rotate in an opposed second direction to reduce a set dose and unstrain the drive spring correspondingly, and
the actuation member being a release member adapted to release the strained drive spring to expel a final set dose amount,
wherein:
when in a mounted state, the add-on actuation member is adapted to actuate the device actuation member when moved from the dose setting state to the dose expelling state, the add-on actuation member having a maximum axial travel relative to the add-on housing of 10 mm.

13. An add-on device as in claim 11, wherein the processor structure for a detected first dose pattern is adapted to:
detect a dose setting pause between two consecutive dose setting rotations in the first direction, and
when one or more dose setting pauses are detected: based on the detected amount of rotation calculating a corrected amount of rotation using a third algorithm compensating for a slack-induced error generated corresponding to the first dose setting pattern when one or more dose setting pauses have been detected.

14. An add-on device as in claim 11, wherein:
the drug expelling structure comprises an over-torque mechanism allowing the dose setting member to be rotated further in the first direction when a predetermined maximum dose has been set, and
the processor structure is adapted to detect an over-torque condition and calculate the amount of rotation of the dose setting member relative to the housing corresponding to the set maximum dose.

15. An add-on device as in claim 11, wherein the rotary sensor is in the form of:
(i) a galvanic rotary encoder comprising a circular array of encoder segments, or
(ii) a magnetic sensor comprising at least one magnetometer adapted to measure a magnetic field from a moving magnet.
